(19) United States
(12) Patent Application Publication
(10) Pub. No.: US 2023/0405530 A1
(43) Pub. Date: Dec. 21, 2023

(54) ROTARY FILTER SYSTEM, A ROTARY FILTER DEVICE AND A SEPARATION SYSTEM

(71) Applicant: Levitronix GmbH, Zürich (CH)

(72) Inventor: Reto SCHÖB, Wollerau (CH)

(21) Appl. No.: 18/205,425

(22) Filed: Jun. 2, 2023

(30) Foreign Application Priority Data
Jun. 16, 2022 (EP) 22179441.5

(51) Int. Cl. B01D 63/16

(52) U.S. Cl. CPC B01D 63/16; B01D 2315/02; B01D 2201/304

(57) ABSTRACT

A rotary filter system includes a drive device and a rotary filter device including a filter housing and a filter unit, the filter housing having an inlet and first and second outlets. The filter unit is in the filter housing and completely enclosed by the filter housing. The filter unit has a filter element delimiting a fluid space from a filtrate space, filtrate is discharged from the filtrate space through the first outlet. The filter unit includes a magnetically effective core in a disk-shaped or ring-shaped manner. The drive device has a drive housing in which a stator is disposed to drive the rotation of the filter unit. The stator is a bearing and drive stator which interacts with the magnetically effective core as an electromagnetic rotary drive, so that the filter unit is capable of being magnetically driven without contact and magnetically levitated with respect to the stator.

### CROSS-REFERENCE TO RELATED APPLICATION

This application claims priority to European Application No. 22179441.5, filed Jun. 16, 2022, the contents of which are hereby incorporated by reference.

### BACKGROUND

### Technical Field

The disclosure relates to a rotary filter system for filtering out a filtrate from a fluid and to a separation system for a bioreactor for extracting a substance from a fluid stored in the bioreactor. The disclosure further relates to a rotary filter device for such a rotary filter system.

### Background Information

In the biotechnological and pharmaceutical industries, conventional bioreactors are frequently used for the recovery of substances, for example proteins, or for the cultivation of cells or other biological material, these bioreactors can be operated both in continuous processes and in batch processes. The operation in continuous processes is usually referred to as perfusion operation and the bioreactor used in this process as a perfusion bioreactor. For example, perfusion processes with bioreactors are thus known, which are used for the continuous cultivation of cells, wherein, for example, metabolic products of the cells are separated by filtration and the cells are returned to the bioreactor. For example, a nutrient solution for the cells can be continuously fed to the bioreactor, thereby replacing the mass or volume of the filtered-out components.

In these processes, it is a typical method to remove the fluid, for example a cell broth (cell broth), from the bioreactor, feed it to a filter device, and return the retentate to the bioreactor again. The substance to be extracted is then removed as filtrate or permeate from the filter device and discharged. Many methods are known for such filter processes. For example, rotary filter devices are known in which the filter membrane, which delimits the filtrate space from which the filtrate is discharged, rotates about an axial direction, for example at a speed in the range of 100 revolutions per minute. The great advantage of these rotary filter devices is that the filter membrane is much less prone to clotting (clotting), for example by solids contained in the fluid. Due to the rotation of the filter membrane, centrifugal forces are generated which lead to the flinging away of deposits in or on the filter membrane.

### SUMMARY

Since biological activities take place in bioreactors, sterility is of very great importance for many processes. Sterilization of the devices, for example by steam sterilization, is very often a time-consuming and cost-intensive factor. For this reason, there is an increasing tendency today to design components of the device as single-use parts for such processes with bioreactors and especially also with perfusion bioreactors, in order to avoid or reduce to a minimum time-consuming sterilization processes. In particular, those components that come into direct contact with the biological substances during the process are often designed as single-use parts. The term “single use parts” (single use) refers to parts or components that can only be used once in accordance with their intended purpose. After use, the single-use parts are disposed of and replaced for the next application by new. i.e., not yet used, single-use parts.

It has been determined that there are problems with regard to sterility that cannot be solved by using single-use components alone. Thus, for example, dynamic seals, such as shaft seals, are provided in rotary filter devices with which the shaft, which drives the rotation of the filter element, is sealed from the static filter housing in the operating state. On the one hand, such dynamic seals can lead to leakage, both in the sense that substances unintentionally escape from the process into the environment and in the sense that contaminants can enter the process through these seals. On the other hand, wear or abrasion in the dynamic seals can lead to the fact that undesired foreign substances enter the process and lead to contamination in the biological processes, which can even endanger the usability of the intended end product.

It is therefore an object of the disclosure to propose a rotary filter system for filtering out a filtrate from a fluid, which enables higher operational reliability with regard to leakage and thus also with regard to sterility. Furthermore, it is an object of the disclosure to propose a rotary filter device for such a rotary filter system. It is a further object of the disclosure to propose a separation system for a bioreactor, in which such a rotary filter system serves to extract a substance from a fluid that is stored in the bioreactor.

The subject matters of the disclosure meeting these object are characterized by the features described herein.

According to the disclosure, a rotary filter system for filtering out a filtrate from a fluid is thus proposed, having a rotary filter device and a drive device, wherein the rotary filter device comprises a stationary filter housing and a filter unit rotatable about an axial direction, wherein the filter housing has an inlet for the fluid, a first outlet for discharging the filtrate, and a second outlet for discharging a retentate, wherein the filter unit is arranged in the filter housing and is completely enclosed by the filter housing, wherein the filter unit has a filter element which delimits a fluid space from a filtrate space, wherein the filtrate can be discharged from the filtrate space through the first outlet, wherein the filter unit further comprises a magnetically effective core which is designed in a disk-shaped or ring-shaped manner, and wherein the drive device has a drive housing in which a stator is provided for driving the rotation of the filter unit. The stator is designed as a bearing and drive stator which interacts with the magnetically effective core of the filter unit as an electromagnetic rotary drive, so that the filter unit can be magnetically driven without contact and can be magnetically levitated with respect to the stator.

Due to the fact that the filter unit comprises the magnetically effective core, which interacts with the drive stator as an electromagnetic rotary drive, with which the filter unit can also be magnetically levitated with respect to the stator, there is no longer any need for a rotating shaft which would have to be led out of the filter housing. As a consequence, there is no longer any need for a dynamic seal on the stationary filter housing, which would have to seal the filter housing at the passage of a rotating shaft. Due to the contactless magnetic drive of the rotation of the filter unit and its magnetic levitation with respect to the stator, the filter housing can thus be designed to be considerably tighter and, in particular, without a shaft seal for sealing the interior of the filter housing from the environment. Thus, there is also no risk that fluid will unintentionally escape from the filter housing via a leakage, or that contaminants or other substances will unintentionally enter the filter housing via a leakage at a seal. By dispensing with a dynamic shaft seal on the filter housing, the risk of wear products, such as abrasion, entering the filter housing and causing contamination is also avoided.

In one embodiment, the filter housing is designed to hermetically enclose the filter unit, so that substances such as the fluid, the retentate and the filtrate can be introduced into the filter housing or discharged from the filter housing exclusively through the inlet and the two outlets. Otherwise, the filter housing is hermetically sealed.

According to an embodiment, a non-contact seal is disposed between the rotatable filter unit and the first outlet of the filter housing, wherein the non-contact seal is arranged in the interior of the filter housing. The non-contact seal inside the filter housing seals between the filter element, which rotates in the operating state, and the stationary first outlet through which the filtrate is discharged from the filtrate space. The non-contact seal, which is designed as a labyrinth seal, for example, reduces or minimizes that leakage flow of the fluid that flows from the inlet directly—i.e., without passing through the filter element—into the filtrate space.

In a further measure that pump vanes for conveying the fluid are disposed on the filter unit and adjacent to the non-contact seal, wherein the pump vanes are designed for rotation about the axial direction and are connected to the filter unit in a torque-proof manner. The pump vanes serve to build up a pressure in the vicinity of the non-contact seal, thereby reducing the leakage flow through the non-contact seal. Depending on the embodiment, the pump vanes can additionally serve to drive the circulation of the fluid or retentate. If, for example, the rotary filter system is connected to a bioreactor, the pump vanes can at least contribute to sucking the fluid from the bioreactor through the inlet and recirculating the retentate to the bioreactor through the second outlet. Depending on the embodiment, the pump vanes can also be the only pumping device for circulating the fluid or retentate.

According to an embodiment, blades for generating a transmembrane pressure across the filter element are disposed on an outer side of the filter unit, wherein the blades are designed for rotation about the axial direction and are connected to the filter unit in a torque-proof manner. These blades serve to adjust or increase the transmembrane pressure across the filter element, i.e., the pressure difference between the fluid space and the filtrate space. For example, the blades can be arranged on the radially outer circumferential surface of the filter unit and/or on an end face of the filter unit.

In an embodiment, the filter housing has an end face at which the inlet, the first outlet and the second outlet are arranged. Thus, all fluid openings of the filter housing, namely the inlet and the two outlets, are disposed at the same end face of the filter housing. This embodiment is particularly advantageous if the drive housing has a centrally arranged cavity, for example a pot-shaped or cup-shaped cavity, into which the filter housing is inserted.

According to an embodiment, the filter unit comprises two magnetically effective cores, each of which is designed in a disk-shaped or ring-shaped manner, and which are arranged at a distance from each other with respect to the axial direction, wherein two stators for driving the rotation of the filter unit are provided in the drive housing of the drive device, wherein each stator is designed as a bearing and drive stator which interacts with one of the magnetically effective cores of the filter unit in each case as an electromagnetic rotary drive, so that the filter unit can be magnetically driven without contact and can be magnetically levitated with respect to the stators. Thus, two drive and bearing points are provided for the drive of the rotation and for the magnetic levitation of the filter unit, which are distanced with respect to the axial direction. In doing so, on the one hand, a higher torque can be generated for the drive of the filter element, and on the other hand, the magnetic levitation and in particular the stabilization against tilting can be improved. In one embodiment, the two magnetic cores are arranged coaxially and thus also parallel to each other. Furthermore, it is advantageous to make the distance between the two magnetically effective cores as large as possible with respect to the axial direction, i.e., to arrange the magnetically effective cores on or in the vicinity of the two end faces which limit the filter unit with respect to the axial direction.

In an embodiment, the rotary filter device and the drive device are designed such that the stationary filter housing can be inserted into the drive housing, and each electromagnetic rotary drive is designed as an internal rotor. In embodiments with only one magnetically effective core in the filter unit, the stator is then arranged in the drive housing in such a way that it surrounds the magnetically effective core radially outwardly when the filter housing is inserted into the drive housing. In embodiments with two magnetically effective cores in the filter unit, the stators are then arranged in the drive housing in such a way that each stator surrounds in each case one of the magnetically effective cores radially outwardly when the filter housing is inserted into the drive housing.

In another embodiment, the rotary filter device and the drive device are designed such that the drive housing can be inserted into a central recess of the stationary filter housing, and each electromagnetic rotary drive is designed as an external rotor. In embodiments with only one magnetically effective core in the filter unit, the stator is then arranged in the drive housing in such a way that the magnetically effective core surrounds the stator radially outwardly when the drive housing is inserted into the filter housing. In embodiments with two magnetically effective cores in the filter unit, the stators are then arranged in the drive housing in such a way that each magnetically effective core surrounds in each case one of the stators radially outwardly when the drive housing is inserted into the filter housing.

With regard to sterility, it is generally preferred that the rotary filter device is designed as a single-use device for single use. Thus, the rotary filter device can only be used exactly once in accordance with their intended purpose and must be replaced for the next application by a new, unused rotary filter device. The drive device is preferably designed as a reusable device for multiple use, wherein the filter housing can be inserted into the drive housing, or the filter housing has a central recess for receiving the drive housing. Since the drive device contains only components which do not come into direct physical contact with the fluid, with the retentate or with the filtrate, sterility or sterilization of the drive device can normally be dispensed with.

A rotary filter device for a rotary filter system is further proposed by the disclosure, which is designed according to the disclosure, wherein the filter housing can be inserted into the drive housing, or the filter housing has a central recess for receiving the drive housing. Here, the rotary filter device usually represents consumable material, while the drive device comprises the reusable components for the drive and the magnetic levitation of the filter unit in the filter housing.

In one embodiment, the rotary filter device is designed as a single-use device for single use.

Furthermore, a separation system for a bioreactor for extracting a substance from a fluid stored in the bioreactor is proposed by the disclosure, wherein the separation system comprises a rotary filter system, which has an inlet for the fluid, a first outlet for discharging a filtrate, and a second outlet for discharging a retentate, wherein a first flow connection is provided with which the inlet can be connected to the bioreactor, wherein a second flow connection is provided with which the second outlet can be connected to the bioreactor, wherein the substance can be removed as the filtrate through the first outlet, and wherein a pumping device is provided for circulating the fluid and the retentate through the first flow connection and the second flow connection. In this embodiment, the rotary filter system is designed according to the disclosure.

According to an embodiment, the pumping device is integrated into the rotary filter system. Here, it is possible that the pumping device is the only device for conveying or circulating the fluid and the retentate. However, embodiments are also possible in which one or more pump(s) are additionally disposed in the first and/or in the second flow connection, which are not an integral part of the rotary filter system.

Preferably, the pumping device is disposed on the rotatable filter unit.

Further advantageous measures and embodiments of the disclosure will become apparent from the disclosure.

### BRIEF DESCRIPTION OF THE DRAWINGS

In the following, embodiments of the invention will be explained in more detail with reference to embodiments and with reference to the drawings.

FIG. 1 is a schematic representation of a first embodiment of a rotary filter system according to the invention,

FIG. 2 is a schematic representation of a second embodiment of a rotary filter system according to the invention.

FIG. 3 is a section through one of the stators of the second embodiment in a section perpendicular to the axial direction along the section line III-III in FIG. 2,

FIG. 4-FIG. 6 are different variants for the first embodiment.

FIG. 7 is a variant for the second embodiment.

FIG. 8 is a schematic representation of a third embodiment of a rotary filter system according to the invention,

FIG. 9 is a variant for the third embodiment,

FIG. 10 is a schematic representation of a fourth embodiment of a rotary filter system according to the invention,

FIG. 11-FIG. 12 are two variants for the fourth embodiment,

Fig. is a schematic representation of an embodiment of a separation system according to an embodiment of the invention for a bioreactor, and

FIG. 14 is a variant for the embodiment of FIG. 12.

### DETAILED DESCRIPTION

FIG. 1 shows in a schematic representation a first embodiment of a rotary filter system according to the disclosure which is designated in its entirety with the reference sign 1. The rotary filter system 1 serves for filtering out a filtrate from a fluid. The fluid which is fed to the rotary filter system 1 is indicated by the arrow F and the filtrate, which is also designated as permeate, is indicated by the arrow P. The arrow R indicates a retentate. The rotary filter system 1 comprises a rotary filter device 10 and a drive device 100. The rotary filter device 10 comprises a stationary filter housing 2, which is stationary in the operating state, i.e., does not rotate. A filter unit 3 rotatable about an axial direction A is disposed in the filter housing 2 which filter unit is completely enclosed by the filter housing 2. In the operating state, the filter unit 3 rotates inside the stationary filter housing 2. The desired axis of rotation about which the filter unit 3 is to rotate defines the axial direction A. A direction perpendicular to the axial direction A is designated as the radial direction.

The filter unit 3 is designed to be essentially cylindrical and has three outer surfaces, namely two end surfaces 31, which delimit the filter unit 3 with respect to the axial direction A, and a circumferential surface 32, which delimits the filter unit 3 with respect to the radial direction.

The filter housing 2 comprises an inlet 23 for introducing the fluid F into the filter housing 2, a first outlet 21 for discharging the filtrate P from the filter housing 2, and a second outlet 22 for discharging the retentate R from the filter housing 2.

The filter unit 3 comprises at least one filter element 4 which delimits a fluid space 41 in the filter housing 2 from a filtrate space 42 in the filter housing 2. The filtrate space 42 is arranged radially inwardly with respect to the filter element 4, and the fluid space 41 is arranged radially outwardly with respect to the filter element 4. In the first embodiment, the filter element 4 extends along the circumferential surface 32 of the filter unit 3 and along one of the two end faces 31 of the filter housing 2, namely along the lower end face 31 according to the representation in FIG. 1.

The first outlet 21 is designed such that it is in flow communication with the filtrate space 42, so that the filtrate P from the filtrate space 42 can be discharged from the filter housing 2 through the first outlet 21.

Any filter element known per se is suitable as filter element 4, although the choice of a suitable filter element 4 of course depends on the particular application.

In the operating state, the filter element 4 rotates about the axial direction A, for example at a rotational speed in the range of 100 rpm (revolutions per minute). The fluid F is introduced into the fluid space 41 through the inlet 23. Those components of the fluid F for which the filter element 4 is permeable pass through the filter element 4 into the filtrate space 42, as the arrows without reference signs indicate, and are then discharged as filtrate P from the filtrate space 42 through the first outlet 21. Those components of the fluid F which cannot penetrate or flow through the filter element 4 remain in the fluid space 41 and are then discharged as retentate R from the filter housing 2 through the second outlet 22. The rotation of the filter element 4 has the effect that deposits on or in the filter element 4 are flung outward by centrifugal forces, so that clotting or clogging of the filter element 4 is effectively prevented or at least drastically reduced.

Since the filter element 4 is disposed on the circumferential surface 32 and on one of the end faces 31 in the first embodiment, the components for which the filter element 4 is permeable can penetrate or flow through the filter element 4 both in the axial direction A and in the radial direction.

The filter unit 3 further comprises a magnetically effective core 6 which is designed in a disk-shaped or ring-shaped manner. The magnetically effective core 6 is arranged on one of the two end faces 31, here on the upper end face 31 according to the representation (according to the representation in FIG. 1). The magnetically effective core 6 comprises one or more permanent magnets 61 (see FIG. 4, for example) for magnetically driving the filter unit 3 to rotate about the axial direction A in a contactless manner, and for magnetically levitating the filter unit 3, preferably in a contactless manner.

Those ferromagnetic or ferrimagnetic materials which are hard magnetic, that is which have a high coercive field strength, are typically called permanent magnets. The coercive field strength is a magnetic field strength which is required to demagnetize a material. Within the framework of this disclosure, a permanent magnet is understood as a material which has a coercive field strength, more precisely a coercive field strength of the magnetic polarization, which amounts to more than 10,000 A/m. All permanent magnets of the magnetically effective core 6 preferably consist of neodymium-iron-boron (NdFeB) or samarium-cobalt (SmCo) alloys.

The magnetically effective core 6 can further comprise a reflux, for example a ring-shaped reflux, which is designed to be continuous and made of a soft magnetic material. For example, each permanent magnet is arranged on the radially inner side of the reflux so that the reflux encloses the permanent magnet or permanent magnets. The reflux guides the magnetic flux and thus serves both to the generation of the torque to drive the rotation of the filter unit 3 and to the magnetic levitation of the filter unit 3. Suitable soft magnetic materials are for example ferromagnetic or ferrimagnetic materials, i.e., in particular iron, nickel-iron or silicon iron.

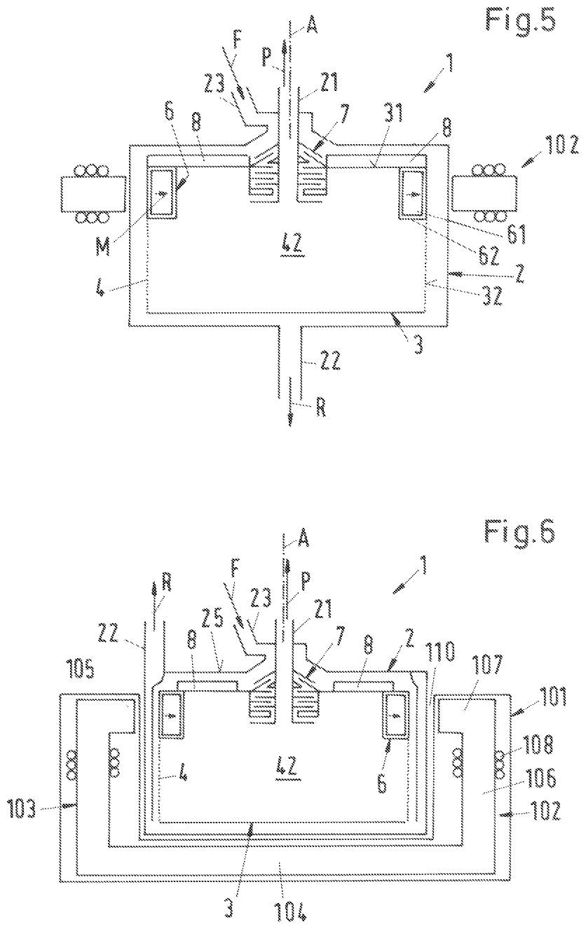

The drive device 100 comprises a drive housing 101 (see FIG. 6, for example), which is not represented in the schematic representation in FIG. 1. A stator 102 is disposed in the drive housing 101 for driving the rotation of the filter unit 3. The stator 102 is designed as a bearing and drive stator that interacts with the magnetically effective core 6 of the filter unit 3 as an electromagnetic rotary drive. With the bearing and drive stator 102, the filter unit 3 can be magnetically driven without contact for rotation about a desired axis of rotation and can be magnetically levitated without contact with respect to the stator 102. In this case, the electromagnetic rotary drive is designed as an internal rotor. i.e., the stator 102 is arranged radially outwardly around the filter housing 2 so that the stator 102 surrounds the magnetically effective core 6 of the filter unit 3. The desired axis of rotation designates that axis about which the filter unit 3 rotates in the operating state when the filter unit 3 is in a centered and non-tilted position with respect to the stator 102.

Preferably, the drive housing 101 comprises a centrally arranged cavity 110 (FIG. 6) into which the filter housing 2 can be inserted. Here, the cavity 110 is preferably dimensioned such that the distance between the stator 102 and the magnetically effective core 6 in the radial direction is as small as possible. This embodiment with the cavity 110 is also particularly advantageous if the filter housing 2 with the filter unit 3 arranged therein is designed as a single-use part for single use. Then, the filter housing 2 can be inserted into the drive device 100 or separated from the drive device 100 in a very simple manner and preferably also without the use of a tool.

The stator 102 comprises a plurality of coil cores 103, for example six coil cores 103, which are connected to each other via a ring-shaped or disk-shaped reflux 104. The coil cores 103 and the reflux 104 are made of a soft magnetic material. Each coil core 103 has a pronounced stator pole 105, wherein the magnetically effective core 6 is arranged between the stator poles 105 in such a way that the pronounced stator poles 105 face the magnetically effective core 6 radially outwardly and are arranged around the magnetically effective core 6.

In the first embodiment, the electromagnetic rotary drive is designed as a so-called temple motor. Each coil core 103 has a longitudinal limb 106 in each case extending in an axial direction A, and a transverse limb 107 arranged perpendicular to the longitudinal limb 106 and extending inwardly in a radial direction. The transverse limb 107 is arranged in each case at an axial end of the associated longitudinal limb 106. Each transverse limb 107 forms one of the pronounced stator poles 105. The coil cores 103 are arranged equidistantly on a circular line so that the transverse limbs 107 surround the magnetically effective core 6 of the filter unit 3 when the filter housing 2 is inserted into the drive device 100. A concentrated winding 108 is arranged in each case on each longitudinal limb 106, surrounding the respective longitudinal limb 106. Embodiments are possible in which exactly one concentrated winding 108 is disposed on each longitudinal limb 106. In other embodiments, more than one winding, for example two concentrated windings, can be disposed on each longitudinal limb 106.

The design as a temple motor is a particularly compact and simultaneously efficient embodiment.

The electromagnetic rotating fields required for the magnetic drive and the magnetic levitation of the filter unit 3 are generated with the concentrated windings 108. With the concentrated windings 108, those electromagnetic rotating fields are generated in the operating state with which a torque is effected on the filter unit 3, and with which a transverse force can be exerted on the filter unit 3 in the radial direction which can be adjusted as desired, so that the radial position of the filter unit 3, i.e. its position in the radial plane perpendicular to the axial direction A, can be actively controlled or regulated.

The magnetically effective core 6 of the filter unit 3 refers to the components of the filter unit 3 which interact magnetically with the stator 102 for the torque generation as well as for the generation of the magnetic levitation forces, i.e., for example the permanent magnet 61 or the permanent magnets and the reflux. It is understood that the magnetically effective core 6 is connected to the remainder of the filter unit 3 in a torque-proof manner.

During operation of the electromagnetic rotary drive, the magnetically effective core 6 of the filter unit 3 interacts with the stator 102 preferably according to the principle of the bearingless motor, in which the filter unit 3 can be magnetically driven without contact as the rotor of the electromagnetic rotary drive and can be magnetically levitated without contact with respect to the stator 102, wherein no separate magnetic bearing is provided. For this purpose, the stator 102 is designed as a bearing and drive stator, with which the filter unit 3 can be magnetically driven without contact about the desired axis of rotation in the operating state—i.e., it can be set in rotation—and can be magnetically levitated without contact with respect to the stator 102. In this embodiment, preferably three degrees of freedom of the filter unit 3, namely its position in the radial plane and its rotation, can be actively regulated. With respect to its axial deflection from the radial plane in the axial direction A, the magnetically effective core 6 of the filter unit 3 is passively magnetically stabilized by reluctance forces, i.e., it cannot be controlled. The magnetically effective core 6 of the filter unit 3 is also passively magnetically stabilized with respect to the remaining two degrees of freedom, namely tilting with respect to the radial plane perpendicular to the desired axis of rotation. Thus, the filter unit 3 is passively magnetically levitated or passively magnetically stabilized in the axial direction A as well as against tilting (a total of three degrees of freedom) and actively magnetically levitated in the radial plane (two degrees of freedom) by the interaction of the magnetically effective core 6 with the coil cores 103.

As is generally the case, an active magnetic levitation is also referred to in the framework of this application as one which can be actively controlled or regulated, for example by the electromagnetic rotating fields generated with the concentrated windings 108. A passive magnetic levitation or a passive magnetic stabilization is one that cannot be controlled or regulated. The passive magnetic levitation or stabilization is based, for example, on reluctance forces, which bring the filter unit 3 back again to its desired position when it is deflected from its desired position, e.g., when it is displaced or deflected in the axial direction A or when it is tilted.

A radial levitation or a levitation in a radial manner refers to a levitation of the filter unit 3 with which the radial position of the filter unit 3 can be stabilized, i.e., a levitation which levitates the filter unit 3 in the radial plane and thus with respect to its radial position.

An axial levitation or a levitation in an axial manner and an axial stabilization or a stabilization in an axial manner, respectively, refers to a levitation or a stabilization of the filter unit 3 with which, on the one hand, the position of the filter unit 3 is stabilized with respect to the axial direction A and with which, on the other hand, the filter unit 3 is stabilized against tilting. Such tilts represent two degrees of freedom and designate deflections in which the momentary axis of rotation of the filter unit 3 no longer points exactly in the axial direction A but encloses an angle different from zero with the desired axis of rotation.

In the case of a bearingless motor, in contrast to classical magnetic bearings, the magnetic levitation and the drive of the motor is realized by electromagnetic rotating fields. Typically, in the bearingless motor, the magnetic drive and levitation function is generated by the superposition of two magnetic rotating fields, which are usually designated as the drive and control fields. These two rotating fields generated with the windings 108 of the stator 102 usually have a pole pair number that differs by one. In this case, tangential forces acting on the magnetically effective core 6 in the radial plane are generated by the drive field, causing a torque, which causes the rotation about the axial direction A. Due to the superposition of the drive field and the control field, it is also possible to generate a transverse force on the magnetically effective core 6 in the radial plane which can be adjusted as desired, with which the position of the magnetically effective core 6 in the radial plane can be regulated. Thus, it is not possible to divide the electromagnetic flux generated by the concentrated windings 108 into an (electro-) magnetic flux that only provides for driving the rotation and an (electro-) magnetic flux that only realizes the magnetic levitation.

This fact that the drive function and the bearing function cannot be separated from each other is what gives the principle of the bearingless motor its name.

To generate the drive field and the control field, it is possible on the one hand to use two different winding systems, namely one for the generation of the drive field with a drive current and one for the generation of the control field with a control current. On the other hand, however, it is also possible to generate the drive and levitation function with only one single winding system—as in the first embodiment described here. This can be realized in such a way that the values for the drive current and the control current determined in each case by a control device are added or superimposed by calculation—e.g., with the aid of software—and the resulting total current is impressed into the respective concentrated winding 108.

As is represented in FIG. 1, a non-contact seal 7 is disposed in the interior of the filter housing 2, which is arranged between the filter unit 3 rotating in the operating state and the first outlet 21 of the filter housing 2. The first outlet 21 is disposed in the center of the filter housing 2 so that it is arranged around the desired axis of rotation. The first outlet 21 extends with respect to the axial direction A through the upper end face 31 of the filter unit 3 according to the representation (FIG. 1) and through the center of the magnetically effective core 6 into the filtrate space 42, so that the filtrate P can be discharged from the filtrate space 42 through the first outlet 21. The non-contact seal 7 is provided at the passage of the stationary outlet 21 into the filter unit 3 rotating in the operating state. Preferably, the non-contact seal 7 is designed as a gap seal and particularly preferably as a labyrinth seal. The non-contact seal 7 is intended to minimize the leakage flow from the inlet 23 along the first outlet 21 into the filtrate space 42. i.e., the leakage flow of the fluid which bypasses the filter element 4. In fact, it is possible that a part of the fluid F enters the filtrate chamber 42 directly from the inlet 23 without passing through the filter element 4. This leakage should at least be reduced by the non-contact seal 7.

Since the non-contact seal 7 is arranged in the interior of the filter housing 2, the filter housing 2 can be designed to be hermetically sealed in such a way that it hermetically encloses the filter unit 3. The fluid F, the filtrate P and the retentate R can only flow into the filter housing 2 or flow out of the filter housing 2 through the inlet 23 and the two outlets 21, 22. Otherwise, the filter housing 2 is designed to be hermetically sealed. In particular, the stationary filter housing 2 is free of dynamic seals that would have to seal the filter housing at the passage of a rotating shaft. Thus, no dynamic seal is disposed between the interior space of the filter housing 2 and the exterior space outside the filter housing 2.

Preferably, pump vanes 8 for conveying the fluid are disposed on the filter unit 3 and adjacent to the non-contact seal 7. The pump vanes 8 are arranged on the end face 31 through which the first outlet 21 passes. According to the representation in FIG. 1, this is the upper end face 31. The pump vanes are arranged on the outside of the filter unit 3. The pump vanes 8 extend in the radial direction and are arranged around the non-contact seal 7. The pump vanes 8 are connected to the filter unit 3 in a torque-proof manner and thus rotate together with the filter unit 3 about the axial direction A.

The pump vanes 8 serve to build up a pressure at the non-contact seal 7 and thus to reduce the leakage flow through the non-contact seal 7. Depending on the embodiment, the pump vanes 8 can additionally serve to drive the circulation of the fluid or retentate. For example, if the rotary filter system 1 is connected to a bioreactor (see FIG. 13, for example), the pump vanes 8 can at least contribute to sucking in the fluid from the bioreactor through the inlet 23 and recirculating the retentate to the bioreactor through the second outlet 22. However, such embodiments of the pump vanes 8 are also possible that cause an increase in pressure only locally in the interior of the filter housing 2, more precisely in the vicinity of the non-contact seal 7, in order to reduce the leakage flow through the non-contact seal 7.

In the first embodiment represented in FIG. 1, the filter housing 2 has an end face 25 at which the inlet 23, the first outlet 21 and the second outlet 22 are arranged. Thus, all fluid openings of the filter housing 2, namely the inlet 23 and the two outlets 21, 22, are disposed at the same end face 25 of the filter housing 2. This embodiment is particularly advantageous if the electromagnetic rotary drive is designed as an internal rotor and as a temple motor as represented in FIG. 1 and also in FIG. 6. The centrally arranged cavity 110 in the drive housing 101 can then be designed in the shape of a pot or cup, for example, so that the filter housing 2 can be inserted into the drive device 100 or separated from the drive device 101 in a very simple manner.

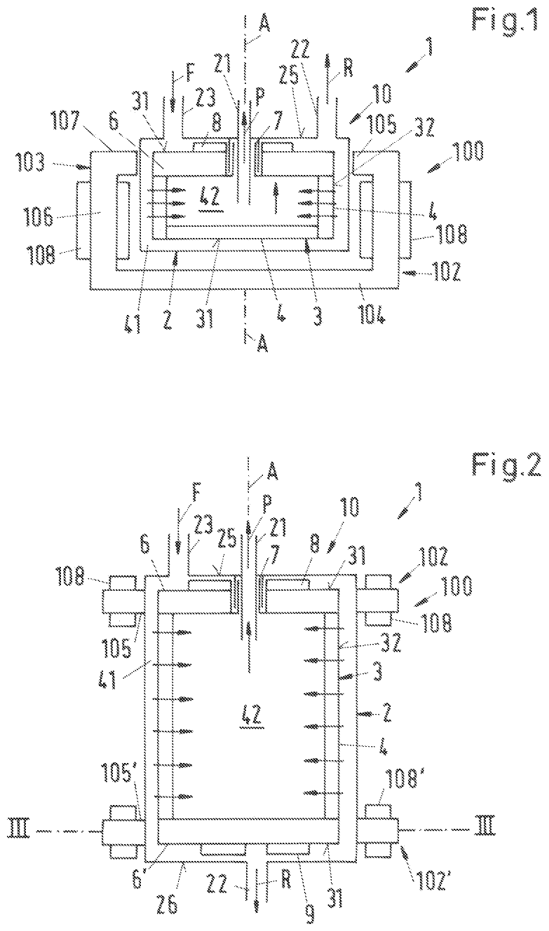

FIG. 2 shows in a schematic representation a second embodiment of a rotary filter system 1 according to the disclosure.

In the following description of the second embodiment, only the differences from the first embodiment will be discussed in more detail. The same parts or functionally equivalent parts of the second embodiment are designated with the same reference signs as in the first embodiment. In particular, the reference signs have the same meaning as already explained in connection with the first embodiment. It is understood that all of the previous explanations of the first embodiment also apply in the same way or in an analogously same way to the second embodiment.

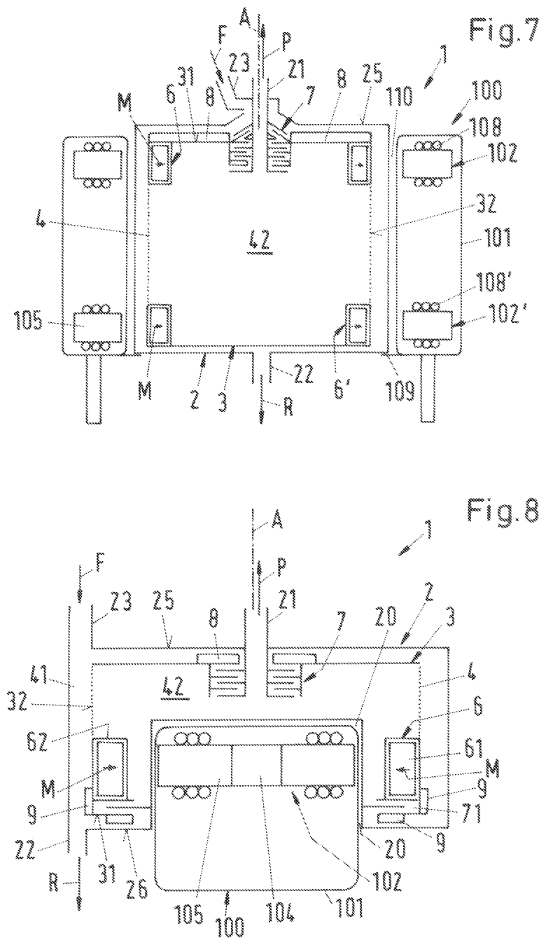

In the second embodiment, the filter unit 3 comprises two magnetically effective cores 6, 6′, each of which is designed in a disk-shaped or ring-shaped manner. Each of the magnetically effective cores 6, 6′ comprises at least one permanent magnet 61 (see FIG. 7, for example). The two magnetically effective cores 6, 6′ are arranged at a distance from each other with respect to the axial direction A. Preferably, one magnetically effective core 6 or 6′ is arranged in each case on each of the two end faces 31 of the filter unit 3, so that the distance between the two magnetically effective cores 31 is as large as possible. The two magnetically effective cores 6, 6′ are arranged coaxially and parallel to each other.

Two stators 102, 102′ are disposed in the drive housing 101 of the drive device 100 for driving the rotation of the filter unit 3. The drive housing 101 is not represented in the schematic representation of FIG. 2. For example, the drive housing 101 is represented in the variant represented in FIG. 7.

Each stator 102, 102′ is designed as a bearing and drive stator, which interacts with one of the magnetically effective cores 6, 6′ of the filter unit 3 in each case as an electromagnetic rotary drive, so that the filter unit 3 can be magnetically driven for rotation without contact and can be magnetically levitated with respect to the stators 102, 102′, preferably magnetically levitated without contact.

Preferably, each of the two electromagnetic rotary drives, each comprising one of the stators 102, 102′ and one of the magnetically effective cores 6, 6′, is designed according to the principle of the bearingless motor described above.

Each of the two electromagnetic rotary drives is designed as an internal rotor. The one of the two stators 102 is arranged radially outwardly around the one of the magnetically effective cores 6, and the other of the two stators 102′ is arranged radially outwardly around the other magnetically effective core 6′.

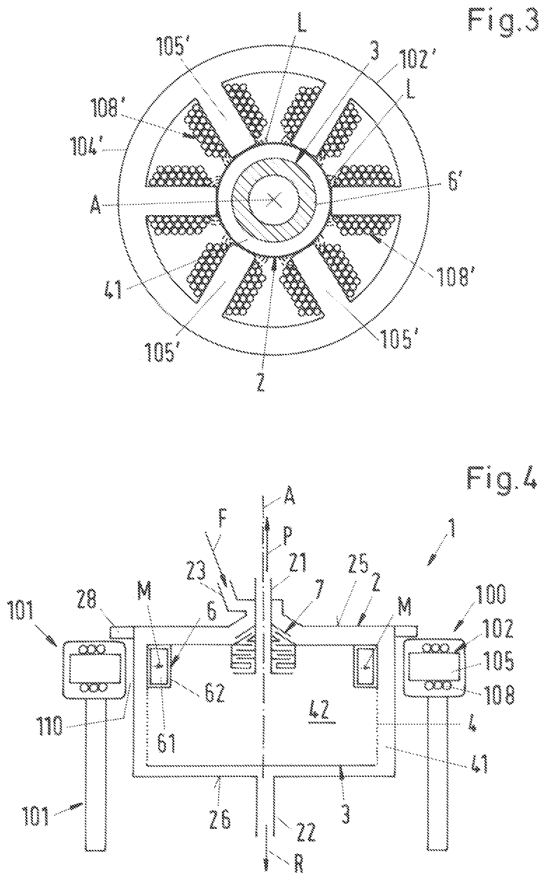

For better understanding, FIG. 3 also shows a section through one of the stators 102′ of the second embodiment in a section perpendicular to the axial direction along the section line III-III in FIG. 2. FIG. 3 shows the lower stator 102′ according to the representation in FIG. 2, wherein the other stator 102 is designed in an analogously same manner.

In the second embodiment, the electromagnetic rotary drives are not designed as a temple motor. The ring-shaped magnetically effective core 6′ of the filter unit 3 is surrounded by the radially outwardly arranged stator 102′. The stator 102′ comprises a plurality of pronounced stator poles 105′—in this case six stator poles 105′—each extending in each case inwardly in the radial direction from the radially outwardly located ring-shaped reflux 104′ toward the filter housing 2 with the magnetically effective core 6′ arranged therein. Each stator pole 105′ is arranged in the radial plane in which the magnetically effective core 6′ is levitated and driven in the operating state. During operation of the electromagnetic rotary drive, the desired position is that the magnetically effective core 6′ is centered between the stator poles 105′.

In order to generate the electromagnetic rotating fields necessary for the magnetic drive and the magnetic levitation of the filter housing 3, the stator poles 105′ carry the concentrated windings 108′. Exactly one concentrated winding 108′ is wound in each case around each stator pole 105′, so that each concentrated winding 108′ is also arranged in the radial plane. In other embodiments, more than one winding, for example two windings, can be disposed in each case on each stator pole 105′.

In FIG. 3, field lines L of the electromagnetic field are also indicated, with which the filter unit 3 is driven or levitated.

Also in the second embodiment, the filter housing 2 is also designed to be hermetically sealed in such a way that it hermetically encloses the filter unit 3. The fluid F, the filtrate P and the retentate R can only flow into the filter housing 2 or flow out of the filter housing 2 through the inlet 23 and the two outlets 21, 22. Otherwise, the filter housing 2 is designed to be hermetically sealed.

In the second embodiment, the inlet 23 for the fluid F and the first outlet 21 for discharging the filtrate P from the filtrate space 42 are disposed at the upper end face 25 of the filter housing 2 according to the representation (FIG. 2). The second outlet 22 for discharging the retentate R from the filter housing 2 is disposed at the other end face 26 of the filter housing 2, i.e., at the lower end face 26 according to the representation in FIG. 2. The second outlet 22 is arranged centrally in the middle of the other end face 26.

Since in the second embodiment one of the two magnetic cores 6, 6′ is arranged in each case on both end faces 31 of the filter unit 3, the filter element 4 extends only along the circumferential surface 32 of the filter unit 3. Those components for which the filter element 4 is permeable penetrate or flow through the filter element 4 only in the radial direction from the outside to the inside, as indicated by the arrows without reference signs in FIG. 2.

As a further option, which can of course also be provided in the first embodiment, a plurality of blades 9 are disposed on at least one outer side 31, 32 of the filter unit 3 for generating a transmembrane pressure across the filter element 4. The blades 9 are designed for rotation about the axial direction A and are connected to the filter unit 3 in a torque-proof manner. As FIG. 2 shows, the blades 9 can be arranged, for example, on the outside of the lower end face 31 of the filter unit 3 according to the representation. Each blade 9 extends outwardly in the radial direction.

The embodiment of the rotary filter system 1 with two electromagnetic rotary drives. i.e. with the two bearing and drive stators 102, 102′ and with the two magnetically effective cores 6, 6′, has the advantage that, on the one hand, a stronger torque can be generated for driving the rotation of the filter unit 3, and that, on the other hand, the magnetic levitation of the filter unit 3 is more stable and can be subjected to greater loads.

Preferably, the two electromagnetic rotary drives are at least substantially identically designed. However, it should be noted that the two electromagnetic rotary drives, even though they are at least substantially identical in design, do not have to be operated in an identical manner when the rotary filter system 1 is in the operating state. Thus, for example, it is possible to generate the torque that drives the rotation of the filter unit 3 using only one of the two electromagnetic rotary drives, and to use the other of the two electromagnetic rotary drives only for the generation of levitating forces for the contactless magnetic levitation of the filter unit 3, so that a torque that drives the rotation of the filter unit 3 is generated only at one of the two magnetically effective cores 6, 6′. Preferably, the levitating forces are generated at both magnetically effective cores. Of course, it is also possible that a torque is generated in each case with both electromagnetic rotary drives, which drives the rotation of the filter unit 3, i.e., that a torque is generated at both magnetically effective cores 6, 6′. It is understood that in this case the torques impressed on the two magnetically effective cores 6, 6′ for driving the filter unit 3 can be the same or different in amount.

With reference to FIG. 4 to FIG. 6, some variants will now be explained which are particularly suitable for the first embodiment (FIG. 1), i.e., for such embodiments in which only one magnetically effective core 6 and only one stator 102 and thus only one electromagnetic rotary drive is provided.

In the variant shown in FIG. 4, the stator 102 is designed in a ring-shaped manner, namely in analogously the same manner as explained in connection with the second embodiment and represented in FIG. 3. The concentrated windings 108 are all arranged in the radial plane in which the magnetically effective core 6 is levitated and driven. The stator 102 is arranged in the drive housing 101, which is designed as a hermetically sealed drive housing 101.

The centrally arranged cavity 110 of the drive housing 101, into which the filter housing 2 can be inserted, is designed as a continuous central opening which extends completely through the drive housing 101 in the axial direction A. As an alternative, the cavity 110 can also be limited by a bottom in the axial direction. The filter housing 2 comprises a support element 28, with which the filter housing 2 is supported on the drive housing 101. For example, the support element 28 is designed as a radially outward flange which surrounds the filter housing 2 at its outer circumference.

The magnetically effective core 6 of the filter unit 3 comprises a ring-shaped permanent magnet 61, which is arranged on the upper end face 31 of the filter unit 3 according to the representation (FIG. 4). The outer diameter of the ring-shaped permanent magnet 61 corresponds substantially to the outer diameter of the filter unit 3. The permanent magnet 61 is preferably diametrically magnetized, as indicated by the arrows with the reference sign M in FIG. 4.

Preferably, a jacket 62 is further provided with which the magnetically effective core 6 is enclosed and preferably hermetically encapsulated so that the magnetically effective core 6 does not come into contact with the fluid F, the filtrate P or the retentate R.

In the variant represented in FIG. 4, the second outlet 22, through which the retentate R is discharged from the filter housing 2, is arranged in the analogously same way as in the second embodiment (FIG. 2) in the lower end face 26 of the filter housing 2 according to the representation (FIG. 4) and preferably in the center of this end face 26.

The first outlet 21, through which the filtrate P is discharged from the filtrate space 42, is arranged in the center of the upper end face 25 of the filter housing 2 according to the representation (FIG. 4). The inlet 23 for the fluid F is arranged concentrically around the first outlet 21.

The non-contact seal 7 is designed as a labyrinth seal.

In the variant represented in FIG. 5, the pump vanes 8 for conveying the fluid are disposed on the upper end face 31 of the filter unit according to the representation. The pump vanes 8 are designed with a large extension in the radial direction, for example in such a way that they extend with respect to the radial direction up to the circumferential surface 32 of the filter unit 3. On the one hand, the pump vanes 8 serve to reduce leakage through the non-contact seal 7, and on the other hand, the pump vanes 8 generate a sufficient pressure and a sufficient flow to convey the fluid through the rotary filter system 1.

For example, if the rotary filter system 1 is connected to a bioreactor (e.g., see FIG. 13), the pump vanes 8 can at least contribute to sucking the fluid F from the bioreactor through the inlet 23 and recirculating the retentate R to the bioreactor through the second outlet 22. Depending on the embodiment, the pump vanes 8 can also be the only pumping device for circulating the fluid F or the retentate R.

The variant represented in FIG. 6 is advantageous in particular for those embodiments in which the electromagnetic rotary drive is designed as a temple motor. In the variant represented in FIG. 6, both the inlet 23 and the two outlets 21, 22 are arranged in the same end face 25, namely in the upper end face 25 of the filter housing 2 according to the representation. Here, the inlet 23 is arranged concentrically around the centrally arranged first outlet 21 in the same way as in the variant represented in FIG. 5. The second outlet 22 is arranged on the periphery of the end face 25 of the filter housing 2.

Due to the fact that the inlet 23 and the two outlets 21, 22 are arranged in the same end face 25 of the filter housing 2, the cavity 110 in the drive housing 101 can be designed in the shape of a cup or pot, so that the filter housing 2 can be inserted into the drive device 100 in a very simple manner.

In FIG. 7, a variant for the second embodiment (FIG. 2) is shown, i.e., for such embodiments in which two magnetically effective cores 6, 6′ and two stators 102, 102′ and thus two electromagnetic rotary drives are provided in each case.

In the variant represented in FIG. 7, the drive housing 101 in which the two stators 102, 102′ are arranged is designed as a substantially ring-shaped drive housing 101. The centrally arranged cavity 110 of the drive housing 101, into which the filter housing 2 can be inserted, is designed as a continuous central opening which extends completely through the drive housing 101 in the axial direction A. The drive housing 101 comprises a support 109 on which the filter housing 2 can be supported when it is inserted into the drive device 100. For example, the support 109 is designed as a ring-shaped protrusion that extends inwardly into the cavity 110 and on which the filter housing 2 can be supported.

Preferably, the drive housing 101 is designed to be hermetically sealed. The first outlet 21, through which the filtrate P is discharged from the filtrate space 42, is arranged in the center of the upper end face 25 of the filter housing 2 according to the representation (FIG. 7). The inlet 23 for the fluid F is arranged concentrically around the first outlet 21.

The non-contact seal 7 is designed as a labyrinth seal.

In the variant represented in FIG. 7, the pump vanes 8 for conveying the fluid are disposed on the upper end face 31 of the filter unit 3 according to the representation. The pump vanes 8 are designed with a large extension in the radial direction, for example in such a way that they extend with respect to the radial direction up to the circumferential surface 32 of the filter unit 3. On the one hand, the pump vanes 8 serve to reduce leakage through the non-contact seal 7, and on the other hand, the pump vanes 8 generate a sufficient pressure and a sufficient flow to convey the fluid through the rotary filter system 1.

For example, if the rotary filter system 1 is connected to a bioreactor (e.g., see FIG. 13), the pump vanes 8 can at least contribute to sucking the fluid F from the bioreactor through the inlet 23 and recirculating the retentate R to the bioreactor through the second outlet 22. Depending on the embodiment, the pump vanes 8 can also be the only pumping device for circulating the fluid F or the retentate R.

FIG. 8 shows in a schematic representation a third embodiment of a rotary filter system 1 according to the disclosure.

In the following description of the third embodiment, only the differences from the first and second embodiment and their variants will be discussed in more detail.

The same parts or functionally equivalent parts of the third embodiment are designated with the same reference signs as in the first and second embodiment and their variants. In particular, the reference signs have the same meaning as already explained in connection with the first and second embodiment and their variants. It is understood that all of the previous explanations of the first and second embodiment and their variants also apply in the same way or in an analogously same way to the third embodiment.

The third embodiment is in an analogously same way as the first embodiment an embodiment in which only one magnetically effective core 6 and only one stator 102 and thus only one electromagnetic rotary drive is provided. In contrast to the first embodiment, in the third embodiment the electromagnetic rotary drive, which comprises the magnetically effective core 6 and the stator 102, is designed as an external rotor. In the design as an external rotor, the stator 102 is arranged radially inwardly in the filter housing 2, so that the magnetically effective core 6 of the filter unit 3 surrounds the stator 102 arranged in the drive housing 101.

For this purpose, the stationary filter housing 2 comprises a central recess 20 in which the drive housing 101 can be inserted, so that the stator 102 is arranged within the magnetically effective core 6. With respect to the axial direction A, the stator poles 105 are arranged at the same level as the magnetically effective core 6 when the drive housing 101 is inserted into the central recess 20 of the filter housing 2.

The embodiment shown in FIG. 8 with the central recess 20, into which the drive device 100 can be inserted, is also advantageous in particular if the filter housing 2 with the filter unit 3 arranged therein is designed as a single-use part for single use. Then, the drive device 100 can be inserted into the filter housing 2 or separated from the filter housing 2 in a very simple manner, and preferably also without the use of a tool.

The magnetically effective core 6 of the filter unit 3 preferably comprises the ring-shaped permanent magnet 61, which in the third embodiment is preferably arranged on the lower end face 31 of the filter unit 3 according to the representation (FIG. 8). The outer diameter of the ring-shaped permanent magnet 61 corresponds substantially to the outer diameter of the filter unit 3. The permanent magnet 61 is preferably magnetized radially from the outside toward the inside, as indicated by the arrows with the reference sign M in FIG. 8.

In the region of the lower end face 31 according to the representation (FIG. 8), an additional seal 71 is preferably provided for sealing between the filter unit 3 rotating in the operating state and the stationary filter housing 2. The additional seal 71 is also designed as a non-contact seal, preferably as a gap seal and particularly preferably as a labyrinth seal. The additional seal 71 reduces the leakage flow of the fluid directly from the fluid space 41 into the filtrate space 42, i.e., the leakage flow of the fluid which bypasses the filter element 4. The additional seal 71 is arranged in the interior of the filter housing 2, so that the filter housing 2 can also be designed to be hermetically sealed in the third embodiment.

The inlet 23 for the fluid F is arranged at the periphery of the end face 25 of the filter housing 2. The first outlet 21, through which the filtrate P can be discharged from the filtrate space 42, is also disposed on the same end face 25 of the filter housing 2. The first outlet 21 is arranged centrally in the middle of the end face 25 of the filter housing 2. The second outlet 22 for discharging the retentate R is arranged at the periphery of the other end face 26 of the filter housing 2.

Adjacent to the non-contact seal 7, the pump vanes 8 can optionally also be provided in the third embodiment to build up a pressure at the non-contact seal 7 and thus to reduce the leakage flow through the non-contact seal 7.

As a further option, the plurality of blades 9 are disposed on at least one outer side 31, 32 of the filter unit 3 for generating a transmembrane pressure across the filter element 4. The blades 9 are designed for rotation about the axial direction A and are connected to the filter unit 3 in a torque-proof manner. As FIG. 8 shows, the blades 9 can be arranged on the circumferential surface 32 of the filter unit 3 and extend in each case in the axial direction A, and/or the blades 9 can be arranged outwardly on the lower end face 31 of the filter unit 3 according to the representation, adjacent to the additional seal 71. Each of the blades 9 arranged on the end face 31 extends outwardly in the radial direction.

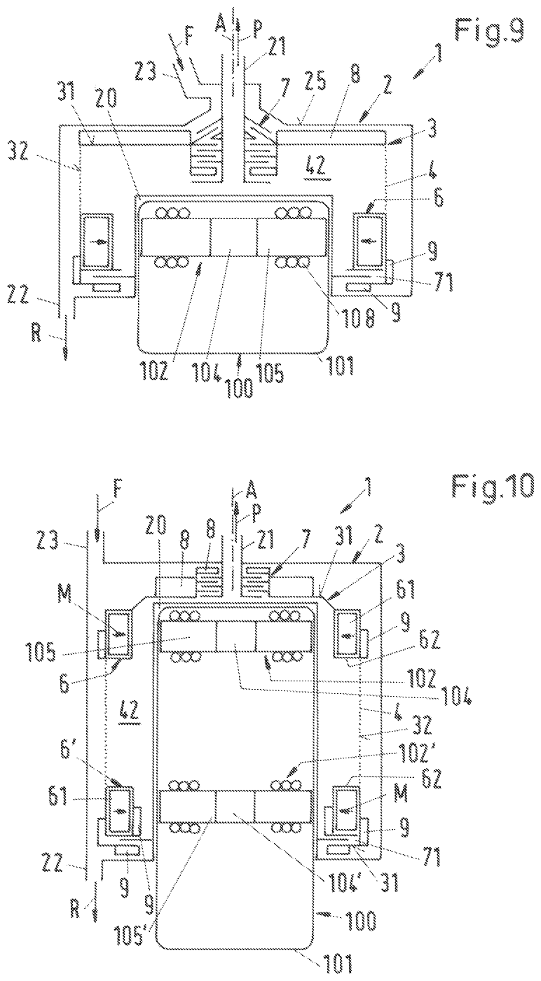

FIG. 9 shows a variant of the third embodiment in which the pump vanes 8 for conveying the fluid are disposed on the upper end face 31 of the filter unit according to the representation. The pump vanes 8 are designed with a large extension in the radial direction so that they can generate a good pumping effect. For example, the pump vanes 8 are designed such that they extend with respect to the radial direction up to the circumferential surface 32 of the filter unit 3. On the one hand, the pump vanes 8 serve to reduce leakage through the non-contact seal 7, and on the other hand, the pump vanes 8 generate a sufficient pressure and a sufficient flow to convey the fluid through the rotary filter system 1.

For example, if the rotary filter system 1 is connected to a bioreactor (e.g., see FIG. 13), the pump vanes 8 can at least contribute to sucking the fluid F from the bioreactor through the inlet 23 and recirculating the retentate R to the bioreactor through the second outlet 22. Depending on the embodiment, the pump vanes 8 can also be the only pumping device for circulating the fluid F or the retentate R.

In the variant represented in FIG. 9, the first outlet 21, through which the filtrate P is discharged from the filtrate space 42, is arranged in the center of the upper end face 25 of the filter housing 2 according to the representation (FIG. 9). The inlet 23 for the fluid F is arranged concentrically around the first outlet 21.

FIG. 10 shows in a schematic representation a fourth embodiment of a rotary filter system 1 according to the disclosure.

In the following description of the fourth embodiment, only the differences from the previously described embodiments and their variants will be discussed in more detail. The same parts or functionally equivalent parts of the fourth embodiment are designated with the same reference signs as in the previously described embodiments and their variants. In particular, the reference signs have the same meaning as already explained in connection with the previously described embodiments and their variants. It is understood that all of the previous explanations regarding the first three embodiments and their variants also apply in the same way or in an analogously same way to the fourth embodiment.

In the fourth embodiment, two electromagnetic rotary drives are provided in an analogously similar way as in the second embodiment. However, these are both designed as external rotors in the fourth embodiment.

For this purpose, the stationary filter housing 2 comprises the central recess 20, into which the drive housing 101 can be inserted.

In the fourth embodiment, the filter unit 3 comprises two magnetically effective cores 6, 6′, each of which is designed in a ring-shaped manner, and each of which optionally has the jacket 62 which encapsulates the respective magnetically effective core 6, 6′. Each of the magnetically effective cores 6, 6′ comprises at least one permanent magnet 61. The two magnetically effective cores 6, 6′ are arranged at a distance from each other with respect to the axial direction A. Preferably, one magnetically effective core 6 or 6′ is arranged in each case on each of the two end faces 31 of the filter unit 3, so that the distance of the two magnetically effective cores 6, 6′ from each other is as large as possible. The two magnetically effective cores 6, 6′ are arranged coaxially and parallel to each other.

Two stators 102, 102′ for driving the rotation of the filter unit 3 are disposed in the drive housing 101 of the drive device 100.

Each stator 102, 102′ is designed as a bearing and drive stator, which interacts in each case with one of the magnetically effective cores 6, 6′ of the filter unit 3 as an electromagnetic rotary drive, so that the filter unit 3 can be magnetically driven without contact for rotation and can be magnetically levitated with respect to the stators 102, 102′, preferably magnetically levitated without contact.

Each of the two electromagnetic rotary drives is designed as an internal rotor. The one of the two magnetically effective cores 6 is arranged radially outwardly around one of the stators 102, and the other of the two magnetically effective cores 6′ is arranged radially outwardly around the other of the two stators 102′.

Each stator 102, 102′ is thus arranged within one of the magnetically effective cores 6, 6. With respect to the axial direction A, the stator poles 105 or 105′ are arranged at the same level as the magnetically effective core 6 or 6′ which surrounds these stator poles 105 or 105′, when the drive housing 101 is inserted into the central recess 20 of the filter housing 2. The stator poles 105 or 105′ extend in each case from the radially inner ring-shaped reflux 104, 104′ in a star shaped manner in radial direction to the outside.

Optionally, pump vanes 8 and/or blades 9 to generate a transmembrane pressure are also provided in the fourth embodiment.

Pump vanes 8 can also be disposed adjacent to the non-contact seal 7 and/or on the non-contact seal. The blades 9 can be provided at one or more of the following locations:
radially outwardly on the circumferential surface 32 of the filter unit 3 at the level (with respect to the axial direction A) of the upper magnetically effective core 6 according to the representation (FIG. 10),
radially outwardly on the circumferential surface 32 of the filter unit 3 at the level (with respect to the axial direction A) of the lower magnetically effective core 6′ according to the representation (FIG. 10),
radially inwardly on the lower magnetically effective core 6′ according to the representation (FIG. 10) and thus adjacent to the additional seal 71,
outside on the lower end face 31 of the filter unit 3 according to the representation (FIG. 10), adjacent to the additional seal 71.

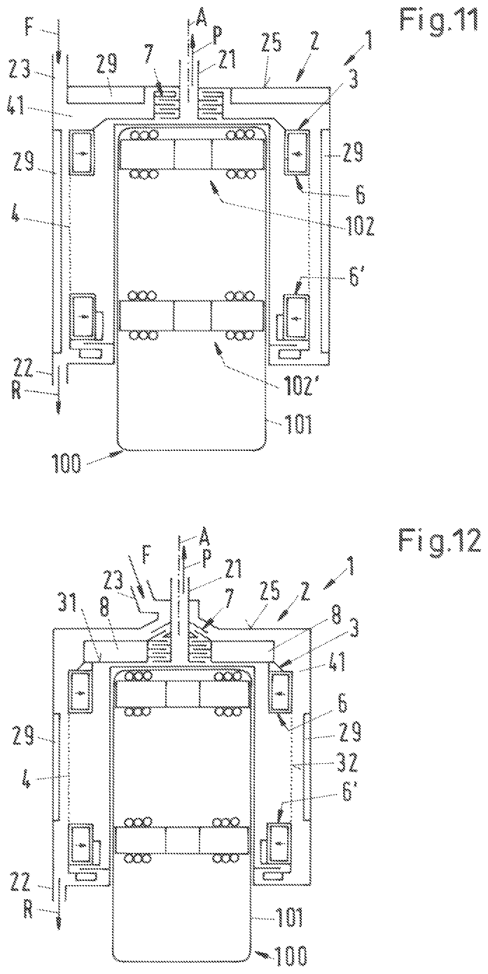

FIGS. 11 and 12 show variants of the fourth embodiment, whereby the variants can also used for the third embodiment.

In the variant represented in FIG. 11, stationary stator blades 29 are disposed in the stationary filter housing 2 for the generation of an additional rotational flow relative to the filter element 4 rotating in the operating state. The stator blades 29 are disposed inside on the wall delimiting the filter housing 2 and extend into the fluid space 41 of the filter housing 2. The stator blades 29 can be arranged on the radially outer wall of the filter housing 2 with a longitudinal extension in the axial direction A. As an alternative or in addition, the stator blades 29 can also be disposed inside on the end face 25 of the filter housing 2, according to the representation (FIG. 11) on the upper end face 25, with a longitudinal extension in the radial direction. The function of the stationary stator blades 29 is to form stagnation zones for the fluid F which remains stationary between the stationary stator blades 29, i.e., a rotational flow of the fluid F in the fluid space 41 relative to the stationary filter housing 2 is at least significantly reduced, if not completely prevented. This leads to a high relative velocity between the filter unit 3 rotating in the operating state and the fluid in the fluid space 41. This high relative velocity is advantageous to prevent or at least reduce clotting of the filter element 4 or deposits on or in the filter element 4.

In the variant represented in FIG. 12, the inlet 23 for the fluid F is arranged concentrically around the first outlet 21, wherein the first outlet 21 again is arranged in the center of the end face 25 of the filter housing 2 so that it is arranged around the desired axis of rotation.

Furthermore, the pump vanes 8 for conveying the fluid F are disposed outside on the upper end face 31 of the filter unit 3 according to the representation (FIG. 12), so that the filter unit 3 has an integrated pumping function. The pump vanes 8 are arranged around the non-contact seal 7, wherein each pump vane 8 extends in the radial direction.

For example, if the rotary filter system 1 is connected to a bioreactor, the pump vanes 8 can at least contribute to sucking the fluid from the bioreactor through the inlet 23 and recirculating the retentate to the bioreactor through the second outlet 22. Depending on the embodiment, the pump vanes 8 can also be the only pumping device between the bioreactor and the rotary filter system 1 for circulating the fluid F or the retentate R. For this purpose, the pump vanes 8 are designed with a large extension in the radial direction, for example in such a way that the pump vanes 8 extend in each case with respect to the radial direction up to the circumferential surface 32 of the filter unit 3. On the one hand, the pump vanes 8 serve to reduce leakage through the non-contact seal 7, and on the other hand, the pump vanes 8 generate a sufficient pressure and a sufficient flow to convey the fluid through the rotary filter system 1.

Preferably, in the rotary filter system 1 according to the disclosure, the rotary filter device 10, which comprises the filter housing 2 with the filter unit 3 arranged therein, is designed as a single-use device for single use, and the drive device 100 is designed as a reusable device for multiple use. For this purpose, it is particularly advantageous that—depending on the embodiment—the filter housing 2 can be inserted into the drive housing 101 and separated from it in a very simple manner, or the drive housing 101 can be inserted into the central recess 20 of the filter housing 2 or removed from it in a very simple manner. Thus, the rotary filter device 10 designed as a single-use part and the drive device 100 designed as a reusable device can be assembled and separated from each other in a very simple manner and preferably without the use of a tool. Then, the rotary filter device 10 represents consumable material as a single-use part, which is used for exactly one application. After this application, the rotary filter device 10 is separated from the drive device 100 and disposed of. For the next application, a new, i.e., unused, rotary filter device 10 is assembled with the drive device 100 to form the rotary filter system 1.

Thus, the rotary filter device 10 represents a separate component of the rotary filter system 1, which can be manufactured and purchased separately from the drive device 100.

Furthermore, a separation system 200 for a bioreactor 300 is proposed by the disclosure. FIG. 13 shows in a schematic representation an embodiment of the separation system 200 according to the disclosure, which is designated in its entirety by the reference sign 200. The bioreactor 300 for which the separation system 200 is suitable is preferably designed as a perfusion bioreactor 300. For example, a perfusion bioreactor 300 is used for continuous cultivation of cells, wherein, for example, metabolic products of the cells or cell-free media are then separated by filtration and the cells are returned into the bioreactor 300. Here, for example, a nutrient solution for the cells can be continuously fed to the bioreactor 300, whereby the mass or the volume of the filtered-out components are replaced. Particularly in the case of such continuously running perfusion processes, it is a particular advantage of the rotary filter system 1 according to the disclosure that the filter housing 2 can be designed as a hermetically sealed filter housing 2 and does not have in particular a dynamic seal which has to seal between the interior of the filter housing 2 and the exterior of the filter housing 2.

The separation system 200 for the bioreactor 300 for extracting a substance from a fluid stored in the bioreactor 300 comprises a rotary filter system 1 which is designed according to the disclosure. The rotary filter system 1 comprises the inlet 23 for the fluid F, the first outlet 21 for discharging the filtrate P, and the second outlet 22 for discharging the retentate R. The filtrate P is the substance to be extracted, for example. The separation system 200 comprises a first flow connection 231, with which the inlet 23 can be connected to the bioreactor 300, and a second flow connection 232, with which the second outlet 22 can be connected to the bioreactor 300, wherein the substance can be removed as the filtrate P through the first outlet 21. Furthermore, a pumping device 241 for circulating the fluid F and the retentate R through the first flow connection 231 and the second flow connection 232 is provided, wherein the pumping device 241 has an inlet 245 and an outlet 246.

For example, the pumping device 241 is designed as a centrifugal pump 241. However, such embodiments are also possible in which the pumping device 241 is integrated into the rotary filter system 1. It is possible to design the pump vanes 8 previously described, which can be disposed on the filter unit 3 of the rotary filter system 1, in such a way that they completely take over the pumping function for circulating the fluid F or the retentate R between the bioreactor 300 and the rotary filter system 1. In such embodiments, the pump vanes 8 then form the pumping device 241. Furthermore, such embodiments are possible in which the pumping device 241 is a separate device, i.e., a device different from the rotary filter system 1. Here, it is possible that only this separate pumping device 241 causes the circulation of the fluid F or the retentate R. i.e., for example, that no pump vanes 8 are provided on the filter unit 3, or that both the separate pumping device 241 is provided and the pump vanes 8, which contribute to the pumping function. Thus, embodiments are possible in which only the separate pumping device 241 is provided for circulation of the fluid F or the retentate R, as well as embodiments in which no separate pumping device 241 is provided and the pumping function is completely taken over by the rotary filter system 1, for example the pump vanes 8 on the filter unit 3, as well as embodiments in which a separate pumping device 241 is provided and the rotary filter system 1 contributes to the pumping function, for example by the pump vanes 8 on the filter unit 3.

In such embodiments in which a separate pumping device 241, i.e., different from the rotary filter system 1, is provided, the pumping device 241 is preferably designed as a centrifugal pump 241, and particularly preferably as a centrifugal pump 241 which is designed according to the principle of the bearingless motor explained above.

Here, the centrifugal pump 241 comprises a rotor for conveying the fluid, and a stator forming an electromagnetic rotary drive with the rotor for rotating the rotor about an axial direction, wherein the rotor comprises a magnetically effective core, and a plurality of vanes for conveying the fluid, wherein the stator is designed as a bearing and drive stator with which the rotor can be magnetically driven without contact and magnetically levitated without contact with respect to the stator. This embodiment of the centrifugal pump 241 with a magnetically levitated rotor, which is simultaneously the pump rotor of the centrifugal pump and the rotor of the electromagnetic rotary drive for driving the rotation, enables an extremely compact, space-saving, and efficient embodiment of the centrifugal pump 241.

According to a particularly preferred embodiment, the centrifugal pump 241 comprises a pump unit having a pump housing, wherein the pump housing comprises an inlet and an outlet for the fluid to be conveyed, wherein the rotor is arranged in the pump housing, and comprises a plurality of vanes for conveying the fluid, and wherein the pump unit is designed in such a way that the pump unit can be inserted into the stator.

Due to the contactless magnetic levitation of the rotor, there is also no need for mechanical bearings, which could lead to contamination of the fluid due to abrasion, for example. The contactless magnetic levitation of the rotor also enables an extremely precise and simple adjustment of the flow generated by the centrifugal pump 241, for example via the rotational speed of the rotor.

Regarding the magnetic levitation of the rotor of the centrifugal pump 241, the rotor is actively magnetically levitated in each case in a radial plane perpendicular to the axial direction, and is passively magnetically stabilized in the axial direction and against tilting.

In particular, the electromagnetic rotary drive of the centrifugal pump 241 can also be designed as a temple motor.

The first flow connection 231 and the second flow connection 232 are preferably realized with pipes that are designed as flexible pipes, i.e., pipes whose walls can be deformed. Each pipe is designed, for example, as a tube, in particular as a plastic tube, which is made for example of a silicone rubber, PVC (polyvinyl chloride). PU (polyurethane), PE (polyethylene). HDPE (high density polyethylene), PP (polypropylene). EVA (ethyl vinyl acetate) or nylon. Preferably, each tube which belongs to the first flow connection 231 or the second flow connection 232 is designed for single use. When designed for single use, those components which contact the substances to be treated, i.e., in this case in particular the tubes, are only used exactly once and are then replaced by new, i.e., unused, single-use parts during the next application.

It is understood that the separation system 200 can comprise further components, such as sensors for detecting pressure or flow or temperature or viscosity. Usually, a control unit (not represented in FIG. 13) is also provided with which the separation system 200 is controlled or regulated.

In the operating state, the fluid F is conveyed by the pumping device 241 from the bioreactor 300 through the first flow connection 231 to the inlet 23 of the rotating filter system 1. The substance to be extracted penetrates the rotating filter element 4 and is subsequently discharged as filtrate P from the filtrate space 42 through the first outlet 21.

The retentate R is conveyed by the pumping device 241 through the second outlet 22 and the second flow connection 232 back into the bioreactor 300.

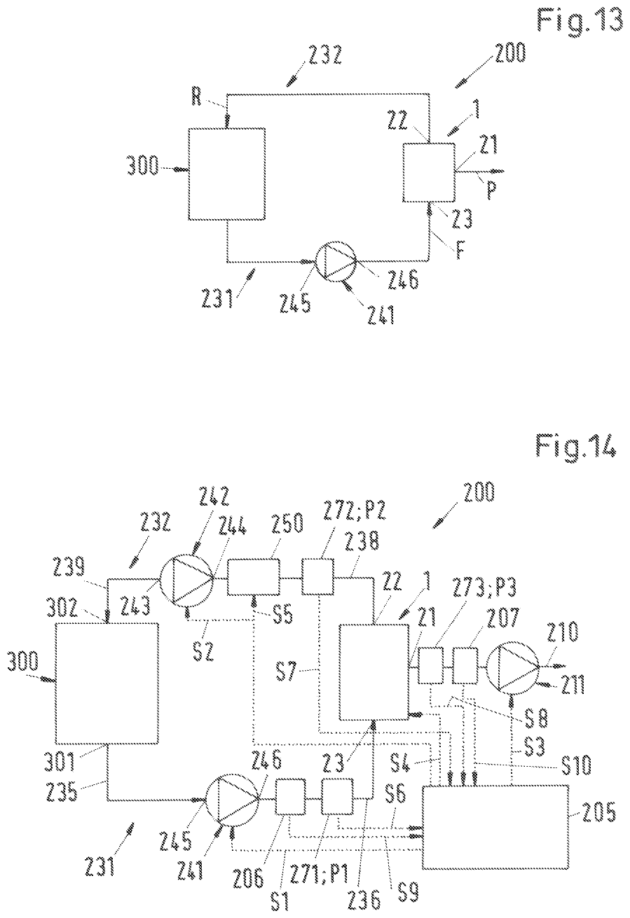

FIG. 14 shows in a schematic representation a variant for the embodiment of the separation system 200 according to the disclosure.

A second centrifugal pump 242 for the fluid F or for the retentate R is arranged in the second flow connection 232, which has an inlet 243 and an outlet 244 for the fluid or the retentate.

Here, the second centrifugal pump 242 is arranged such and is operated such that it operates in the opposite direction to the pumping device 241. This means that the outlet 246 of the pumping device 241 is connected to the outlet 244 of the second centrifugal pump 242 via the rotary filter system 1. Both the outlet 246 of the pumping device 241 and the outlet 244 of the second centrifugal pump 242 are connected in each case to the rotary filter system 1.

Since the second centrifugal pump 242 operates in the opposite direction to the pumping device 241, the second centrifugal pump 242 can create a counter-pressure at the second outlet 22 of the rotary filter system 1 so that the pressure at the second outlet 22 increases. In doing so, the pressure drop increases across the filter element 4, which allows the permeate flow, i.e. the flow through the filter element 4, to be increased. The pressure of the fluid at the second outlet 22 can be adjusted by the second centrifugal pump 242 with high accuracy, in a simple manner, reproducibly and reliably over a wide range of operation.

The pressure of the fluid at the inlet 23 of the rotary filter system 1 is designated as a first pressure P1. The pressure of the retentate at the second outlet 22 is designated as a second pressure P2. The pressure of the filtrate at the first outlet 21 is designated as a third pressure P3. As is common practice, the transmembrane pressure TMP is then defined as:

TMP=(P1+P2)/2−P3

The transmembrane pressure is also designated as transmembrane pressure difference.

In the variant represented in FIG. 14, it is of course also possible that the pumping device 241 is completely integrated into the rotary filter system 1 and is realized there by the pump vanes 8, or that both the separate pumping device 241 outside the rotary filter system 1 and the pump vanes 8 in the rotary filter system 1 are provided for the pumping function.

If the pump vanes 8 are provided for the generation of a pressure in the rotary filter system 1, the pressure generated by the pump vanes 8 adds to the first pressure P1.

As an alternative or in addition to the second centrifugal pump 242, an actuatable control valve 250 can also be disposed in the second flow connection 232 with which the flow rate through the second flow connection 232 can be adjusted. By adjusting the flow rate through the second flow connection 232, the second pressure P2 can also be adjusted. For example, if the flow rate through the second flow connection 232 is reduced while the first pumping device 241 is kept in constant operation, the second pressure P2 at the second outlet 22 of the rotary filter system 1 increases as a result.

The optionally provided control valve 250 can either replace the second centrifugal pump 242 or can be disposed in addition to the second centrifugal pump 242. If, as represented in FIG. 14, both the second centrifugal pump 242 and the control valve 250 are provided, the control valve 250 is arranged between the outlet 244 of the second centrifugal pump 242 and the second outlet 22 of the rotary filter system 1.

The first flow connection 231 comprises a supply tube 235 which connects a first opening 301 of the bioreactor 300 to the inlet 245 of the pumping device 241, and a feeding tube 236 which connects the outlet 246 of the pumping device 241 to the inlet 23 of the rotary filter system 1. If no separate pumping device 241 is provided, the supply tube 235 and the feeding tube 236 can be designed as a structural unit which connects the first opening 301 to the inlet 23 of the rotary filter system 1.

Furthermore, a flow sensor 206 is provided for determining the flow rate of the fluid through the first flow connection 231. For example, the flow sensor 206 is disposed in or on the feeding tube 236 of the first flow connection 231. Of course, it is also possible to provide the flow sensor 206 in or on the supply tube 235, i.e., between the bioreactor 300 and the pumping device 241. In particular, the flow sensor 206 can be designed as a so-called clamp-on sensor, i.e., as a flow sensor 206 that is clamped onto the feeding tube 236 or onto the supply tube 235, so that the feeding tube 236 or the supply tube 235 is clamped in the measuring area of the flow sensor 206.

The second flow connection 232 comprises a discharge tube 238 which connects the second outlet 22 of the rotary filter system 1 to the control valve 250, or if the latter is not provided, to the outlet 244 of the second centrifugal pump 242.

The second flow connection 232 further comprises a return tube 239 which connects the inlet 243 of the second centrifugal pump 242 to a second opening 302 of the bioreactor 300. If the second centrifugal pump 242 is not provided, the return tube 239 connects the second opening 302 of the bioreactor 300 to the control valve 250.

Thus, both the outlet 246 of the pumping device 241 and the outlet 242 of the second centrifugal pump 242 or the control valve 250 are connected in each case to the rotary filter system 1, namely to the inlet 23 or to the second outlet 22 of the rotary filter system 1. For this reason, a counter-pressure can be generated at the second outlet 22 by the second centrifugal pump 242 and/or by the control valve 22, so that the second pressure P2 can be adjusted at the second outlet 22.

In the operating state, the pumping device 241 and/or the pump vanes 8 of the rotary filter system 1 serve to move the fluid through the rotary filter system 1 and via the filter element 4. The pumping device 241 and/or the pump vanes 8 circulate the fluid from the bioreactor 300 through the first flow connection 231, through the rotary filter system 1 and as the retentate through the second flow connection 232 back into the bioreactor 300.

In the operating state, the second centrifugal pump 242 and/or the control valve 250 serve(s) to generate a counter-pressure at the second outlet 22 of the rotary filter system 1, i.e., the second centrifugal pump 242 and/or the control valve 250 is/are operated in such a way that they increase the second pressure P2 prevailing at the second outlet 22.

Preferably, the separation system 200 further comprises a plurality of pressure sensors 271, 272, 273, wherein the pressure sensors 271, 272, and 273 are preferably arranged and designed in such a way that they can be used to determine the transmembrane pressure across the filter element 4.

In the variant represented in FIG. 14, a total of three pressure sensors 271, 272, 273 are provided, namely a first pressure sensor 271 with which the first pressure P1 of the fluid at the inlet 23 of the rotary filter system 1 can be determined, a second pressure sensor 272 with which the second pressure P2 of the retentate at the second outlet 22 of the rotary filter system 1 can be determined, and a third pressure sensor 273 with which the third pressure P3 at the first outlet 21 of the rotary filter system 1 can be determined.

In the variant represented in FIG. 14, the first pressure sensor 271 is disposed between the flow sensor 206 and the inlet 23 of the rotary filter system 1, namely in or on the feeding tube 236. The second pressure sensor 272 is disposed between the second outlet 22 of the rotary filter system 1 and the control valve 250 or the second centrifugal pump 242, namely in or on the discharge tube 238. The third pressure sensor 273 is arranged on or downstream of the first outlet 21 in or on a filtrate pipe 210 through which filtrate P is discharged from the first outlet 21.

Optionally, a second flow sensor 207 is disposed in or on the filtrate pipe 210, by which the flow rate of the filtrate P through the filtrate pipe 210 can be determined.

Optionally, a third pumping device 211 is further disposed in the filtrate pipe 210 in order to convey the filtrate P through the filtrate pipe 210. Preferably, the second flow sensor 207 and the third pressure sensor 273 are arranged upstream of the third pumping device 211. The third pumping device 211 is designed as a peristaltic pump, for example.

Furthermore, a control unit 205 is provided with which the separation system 200 is operated and actuated or controlled. For this purpose, the control unit 205 is signal-connected to the different components of the separation system. In FIG. 14, the signal connections are indicated by dashed arrows and can be designed in each case as physical connections, for example as signal cables or signal lines, or as wireless (wireless) signal connections.

The signal connections S1, S2 and S3 connect the control unit 205 to the first pumping device 241, the second centrifugal pump 242 and the third pumping device 211. The pumping devices 241, 242, 211 are actuated with these signal connections S1, S2. S3, for example the rotational speed or the flow to be generated is controlled or regulated. The signal connection S4 connects the control unit 205 to the rotary filter system 1 and serves, for example, to adjust or regulate the rotational speed of the filter unit 3. The signal connection S5 serves to actuate the control valve 250. The flow rate through the control valve 250 can be adjusted via the signal connection S5. The signal connections S6, S7, S8 serve for data exchange with the pressure sensors 271, 272, 273. The pressure sensors 271, 272, 273 can transmit their respective measured values to the control unit 205 via the signal connections S6. S7. S8 The signal connections S9. S10 serve for data exchange with the flow sensors 206, 207. The flow sensors 206, 207 can transmit their respective measured values to the control unit 205 via the signal connections S9 and S10.

Preferably, but not necessarily, the second centrifugal pump 242 is designed at least substantially identically to the pumping device 241, which—as already mentioned—is preferably also designed as a centrifugal pump 242. In particular, it is therefore preferred that both the pumping device 241 and the second centrifugal pump 242 are each designed as a centrifugal pump 241, 242, which are designed according to the principle of the bearingless motor already explained.

In analogously the same way as already explained for the rotary filter system 1 according to the disclosure, the separation system 200 can also be designed in such a way that it comprises a reusable system which is designed for multiple use and a single-use system which is designed for single use. In this respect, the reusable system comprises in particular those components which do not come into contact with the fluid or the retentate or the filtrate, i.e., in particular the drive device 100 of the rotary filter system 1, the stators of the centrifugal pumps 241, 242 and for example at least parts of the pressure sensors 271, 272, 273. The pressure sensors 271, 272, 273 can be designed in such a way that they comprise in each case single-use parts and reusable parts.

The single-use system comprising the components designed for single use comprises at least the following components: the rotary filter device 10, the pump units for each centrifugal pump 241, 242, a plurality of tubes 235, 236, 238, 239 designed to realize the first flow connection 231 and the second flow connection 232, and optionally at least one tube for the filtrate pipe 210.

It is a further substantial aspect that all parts of the rotary filter system 1 and the separation system 200 which come into contact with the fluid F or the retentate R or the filtrate P, in particular the rotary filter device 10, the flow connections 231, 232, and if applicable the pressure sensors 271, 272, 273 and the pump units of the centrifugal pumps 241 and 242 or their components should be sterilizable for certain applications. It is particularly advantageous if all the components mentioned can be gamma sterilizable. In this type of sterilization, the component to be sterilized is applied with gamma radiation. The advantage of gamma sterilization, for example in comparison with steam sterilization, is in particular that sterilization can also take place through the packaging. For single-use parts in particular, it is a common practice that the parts are placed in the packaging intended for shipping after they are manufactured and then stored for a period of time before being shipped to the customer. In such cases, sterilization takes place through the packaging just before delivery to the customer, which is not possible with steam sterilization or other processes.

With regard to single-use parts, it is generally not necessary for them to be sterilizable more than once. This is a great advantage, particularly in the case of gamma sterilization, because the application of gamma radiation to plastics can lead to degradation, so that multiple gamma sterilization can render the plastic unusable.

Since sterilization under high temperatures and/or under high (steam) pressure can usually be dispensed with for single-use parts, less expensive plastics can be used, for example those that cannot withstand high temperatures or that cannot be subjected to multiple high temperature and pressure levels.

Considering all these aspects, it is therefore preferred to use such plastics that can be gamma-sterilized at least once for the manufacture of single-use parts. The materials should be gamma-stable for a dose of at least 40 kGy to enable a single gamma sterilization. In addition, no toxic substances should be generated during gamma sterilization. In addition, it is preferred that all materials that come into contact with the substances to be mixed or the intermixed substances meet USP Class VI standards.

For example, the following plastics are preferred for manufacturing the parts consisting of plastic, e.g., the filter housing 2 of the rotary filter system 1; polyethylene (PE), polypropylene (PP), low density polyethylene (LDPE), ultra low density polyethylene (ULDPE), ethylene vinyl acetate (EVA), polyethylene terephthalate (PET), poly vinylchloride (PVC), polyvinylidene fluoride (PVDF), acrylonitrile butadiene styrene (ABS), polyacryl, polycarbonate (PC).

Less suitable or even unsuitable materials for manufacturing the plastic parts of the single-use components are, for example, the materials known under the brand name Teflon, polytetrafluoroethylene (PTFE) and perfluoroalkoxy polymers (PFA). In the case of these materials, there is in fact a risk during gamma sterilization that hazardous gases escape, such as fluorine, which can then form toxic or harmful compounds such as hydrofluoric acid (HF).

What is claimed is:

1. A rotary filter system for filtering out a filtrate from a fluid, comprising:
a rotary filter device; and
a drive device,
the rotary filter device comprising a stationary filter housing and a filter unit rotatable about an axial direction, the filter housing having an inlet for the fluid, a first outlet for discharging the filtrate, and a second outlet for discharging a retentate, the filter unit arranged in the filter housing and being completely enclosed by the filter housing, the filter unit having a filter element delimiting a fluid space from a filtrate space, the filtrate capable of being discharged from the filtrate space through the first outlet, the filter unit further comprising a magnetically effective core designed in a disk-shaped or ring-shaped manner, and the drive device having a drive housing in which a stator is disposed to drive the rotation of the filter unit, the stator being a bearing and drive stator configured to interact with the magnetically effective core of the filter unit as an electromagnetic rotary drive, so that the filter unit is capable of being magnetically driven without contact and magnetically levitated with respect to the stator.

2. The rotary filter system according to claim 1, wherein the filter housing is configured to hermetically enclose the filter unit.

3. The rotary filter system according to claim 1, wherein a non-contact seal is disposed between the rotatable filter unit and the first outlet of the filter housing, and the non-contact seal is arranged in an interior of the filter housing.

4. The rotary filter system according to claim 3, wherein a plurality of pump vanes configured to convey the fluid are disposed on the filter unit and adjacent to the non-contact seal, and the pump vanes are configured to rotate about the axial direction and are connected to the filter unit in a torque-proof manner.

5. The rotary filter system according to claim 1, wherein blades configured to generate a transmembrane pressure via the filter element are disposed on an outer side of the filter unit, and the blades are configured to rotate about the axial direction and are connected to the filter unit in a torque-proof manner.

6. The rotary filter system according to claim 1, wherein the filter housing has an end face at which the inlet, the first outlet and the second outlet are arranged.

7. The rotary filter system according to claim 1, wherein the magnetically effective core is one of a first magnetically effective core and the filter unit includes a second magnetically effective core, each of the second magnetically effective core being disk-shaped or ring-shaped, and the first and second magnetically effective cores being arranged at a distance from each other with respect to the axial direction, the stator being a first stator of first and second stators configured to drive rotation of the filter unit are disposed in the drive housing of the drive device, the second stator is a bearing and drive stator configured to interact with one of the second magnetically effective core of the filter unit as an electromagnetic rotary drive, so that the filter unit is capable of being magnetically driven without contact and magnetically levitated with respect to the first and second stators.

8. The rotary filter system according to claim 7, wherein the rotary filter device and the drive device are configured such that the stationary filter housing is capable of being inserted into the drive housing, and each of the electromagnetic rotary drives is an internal rotor.

9. The rotary filter system according to claim 7, wherein the rotary filter device and the drive device are configured such that the drive housing is capable of being inserted into a central recess of the stationary filter housing, and each of the electromagnetic rotary drives is an external rotor.

10. The rotary filter system according to claim 1, wherein the rotary filter device is a single-use device for single use, and the drive device is a reusable device for multiple use, and the filter housing capable of being inserted into the drive housing or the filter housing has a central recess configured to receive the drive housing.

11. A rotary filter device for a rotary filter system, according to claim 1, comprising:
a stationary filter housing; and
a filter unit rotatable about an axial direction,
the filter housing having an inlet for fluid, a first outlet for discharging filtrate, and a second outlet for discharging a retentate, the filter unit arranged in the filter housing and being completely enclosed by the filter housing, the filter unit having a filter element delimiting a fluid space from a filtrate space, the filtrate capable of being discharged from the filtrate space through the first outlet, the filter unit further comprising a magnetically effective core designed in a disk-shaped or ring-shaped manner, the filter housing configured to be inserted into a drive housing, or the filter housing having a central recess configured to receive the drive housing.

12. The rotary filter device according to claim 11, wherein the rotary filter device is a single-use device for single use.

13. A separation system for a bioreactor for extracting a substance from a fluid stored in the bioreactor, the separation system comprising:
a rotary filter system according to claim 1;
a first flow connection with which the inlet is configured to be connected to the bioreactor;
a second flow connection with which the second outlet is configured to connected to the bioreactor, the substance capable of being removed as the filtrate through the first outlet, and
a pumping device configured to circulate the fluid and the retentate through the first flow connection and the second flow connection.

14. The separation system according to claim 13, wherein the pumping device is integrated into the rotary filter system.

15. The separation system according to claim 14, in which the pumping device is disposed on the rotatable filter unit.
